(19) United States
(12) Patent Application Publication
(10) Pub. No.: US 2008/0018734 A1
(43) Pub. Date: Jan. 24, 2008

(54) Endoscope System

(75) Inventor: Noriko IRIYAMA, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/780,571

(22) Filed: Jul. 20, 2007

(30) Foreign Application Priority Data
Jul. 20, 2006 (JP) 2006-198559
Jul. 18, 2007 (JP) 2007-186827

(51) Int. Cl. A61B 1/06

(52) U.S. Cl. 348/72; 600/182

(57) ABSTRACT

An electronic endoscope system, having at least one electronic endoscope, and a processor to regenerate an image captured by the electronic endoscope is provided. The electronic endoscope includes an optical objective system, a first image capturing element with higher sensitivity, which generates a first voltage signal, and a second image capturing element with lower sensitivity, which generates a second voltage signal. The processor includes a threshold setting system, which sets a threshold for judging which of the first voltage signal and the second voltage signal is used to regenerate the image, a judging system, which judges whether the first voltage signal is lower than or equal to the threshold, and an image processing system, which generates a usable signal to be used to regenerate the image based on one of the first voltage signal and the second voltage signal.

### BACKGROUND OF THE INVENTION

The present invention relates to an endoscope system including an electronic endoscope for observing inside a body cavity.

Conventionally, an endoscope system for observing and treating inside a body cavity is widely known to be used. The endoscope system generally includes an electronic endoscope that captures an image inside the body cavity, a processor that processes the image signal obtained by the electronic endoscope, and a monitor in which video signals processed and generated by the processor is displayed. Such an endoscope system is disclosed in U.S. Pat. No. 5,902,230, for example.

In the endoscope system as disclosed in the above-referenced publication, an image is captured by a single image capturing element, and quality of the captured image tends to depend largely on performance of the single image capturing element with a constant sensitivity. Accordingly, when brightness in one observation object is uneven, details in some areas in the captured image of the observation object may be undesirably lost (lost balanced) in white or black. Thus, it tends to be difficult to obtain desirable quality of images.

Further, generally, the endoscopes are suitably exchanged according to the observation object, and such conditions for the lost balance in white and black may vary among different endoscopes. For example, details can be lost in white when relatively wide areas, wherein illumination light can be diffused such as in a stomach and a duodenum, are observed. On the contrary, details can be lost in black when relatively narrow areas with substantial length such as in a large intestine, wherein illumination light cannot be diffused, are observed.

### SUMMARY OF THE INVENTION

In view of the foregoing drawbacks, the present invention is advantageous in that an endoscope system with an electronic endoscope, in which images with improved quality can be suitably obtained regardless of brightness differences in the observation object, is provided. In such an endoscope system, preferable brightness can be maintained in the obtained images regardless of forms of the observation objects.

According to an aspect of the present invention, there is provided an electronic endoscope system, having at least one exchangeable electronic endoscope to capture an image of an observation object, and a processor, which is connectable with the electronic endoscope, to regenerate the image captured by the electronic endoscope. The electronic endoscope includes an optical objective system, a first image capturing element with higher sensitivity, which captures the image of the observation object and generates a first voltage signal representing the image based on the higher sensitivity, and a second image capturing element with lower sensitivity being lower than the sensitivity of the first image capturing element, which captures the image of the observation object and generates a second voltage signal representing the image based on the lower sensitivity. The processor includes a threshold setting system, which sets a threshold for judging which of the first voltage signal and the second voltage signal is used to regenerate the image based on suitability of the electronic endoscope, a judging system, which judges as to whether the first voltage signal obtained from the first image capturing element is either lower than or equal to the threshold set by the threshold setting system, and an image processing system, which generates a usable signal to be used to regenerate the image based on the first voltage signal when the first voltage signal is judged to be either lower than or equal to the threshold and generates the usable signal based on the second voltage signal when the first voltage signal is judged to be higher than the threshold.

Optionally, the electronic endoscope may further include a memory system, which stores information specific to the electronic endoscope, and the threshold setting system reads the information from the memory system of the electronic endoscope being connected with the processor so that the threshold is set based on the information.

Optionally, the information specific to the electronic endoscope may include information concerning the threshold.

Optionally, the electronic endoscope system may include a plurality of the exchangeable electronic endoscopes. Each of the plurality of exchangeable electronic endoscopes may be provided with the information concerning the threshold which is specific to the exchangeable electronic endoscope.

Optionally, the image processing system may divide one frame of the image captured by the electronic endoscope into a plurality of divided areas, generate the usable signal for each divided area based on the first voltage signal when the first voltage signal is judged to be lower than or equal to the threshold and on the second voltage signal when the first voltage signal is judged to be higher than the threshold, and combine the generated usable signals for each divided area to regenerate one frame of the image based on the generated usable signal.

Optionally, the image processing system may adjust the second voltage signal by adding a correction value to the second voltage signal so that an output level of the second voltage signal substantially equals an output level of the first voltage signal at a point where the output level of the first voltage signal substantially equals the threshold. The correction value may be determined based on the suitability of the electronic endoscope.

Optionally, the electronic endoscope may be provided with a beam splitter to split a beam entering the electronic endoscope substantially evenly into two directions so that substantially same amount of light is transmitted to the first image capturing element and the second image capturing element. The beam splitter may include a half mirror, which deflects a part of the beam entering the electronic endoscope toward the first image capturing element and transmits the remaining part of the beam to the second image capturing element.

Optionally, the first image capturing element and the half mirror may be integrally attached to a driving system. The first image capturing element and the half mirror may be driven by the driving system which is slidable in parallel with an optical axis of the objective optical system so that focusing in the first image capturing element is adjusted.

Optionally, the image processing system may generate a first image signal based solely on the first voltage signal obtained from the first image capturing element and generate a second image signal based solely on the second voltage signal obtained from the second image capturing element.

Optionally, the electronic endoscope system may include a displaying device, which displays the image regenerated by the processor on a screen. The displaying device may be capable of simultaneously displaying at least one of an image corresponding to the usable signal, an image corresponding to the first image signal, and an image corresponding to the second image signal on the screen.

### BRIEF DESCRIPTION OF THE DRAWINGS

FIG. 1 is a schematic diagram of an endoscope system according to a first embodiment of the present invention.

FIG. 2 is a partially enlarged cross-sectional view of an electronic endoscope according to the first embodiment of the present invention.

FIG. 3 is a chart showing correspondence between amounts of light and output signal levels in a first image capturing element according to the first embodiment of the present invention.

FIG. 4 is a chart showing correspondence between amounts of light and output signal levels in a second image capturing element according to the first embodiment of the present invention.

FIG. 5 is a chart showing correspondence between amounts of light and output signal levels in the first and second image capturing elements according to the first embodiment of the present invention.

FIG. 6 is a flowchart to illustrate a signal modifying process to be executed by the processor according to the first embodiment of the present invention.

FIG. 7 is a chart showing correspondence between amount of light and output signal level of usable signals generated by a signal process circuit according to the first embodiment of the present invention.

FIG. 8 is a chart showing correspondence between amounts of light and output signal levels in three image capturing elements according to a second embodiment of the present invention.

FIG. 9 is a cross-sectional partial view of an electronic endoscope with three image capturing elements having different sensitivities according to a second embodiment of the present invention.

FIG. 10 is a chart showing correspondence between amount of light and output signal level in the third image capturing element according to the second embodiment of the present invention.

FIG. 11 is a chart showing correspondence between amount of light and output signal level of usable signals generated by the signal process circuit according to the second embodiment of the present invention.

FIG. 12 is a chart showing correspondence between amounts of light and the signal levels of the usable signals generated in the signal process circuit according to the second embodiment of the present invention.

### DETAILED DESCRIPTION OF THE EMBODIMENTS

Hereinafter, referring to the accompanying drawings, according to illustrative embodiments of the invention will be described.

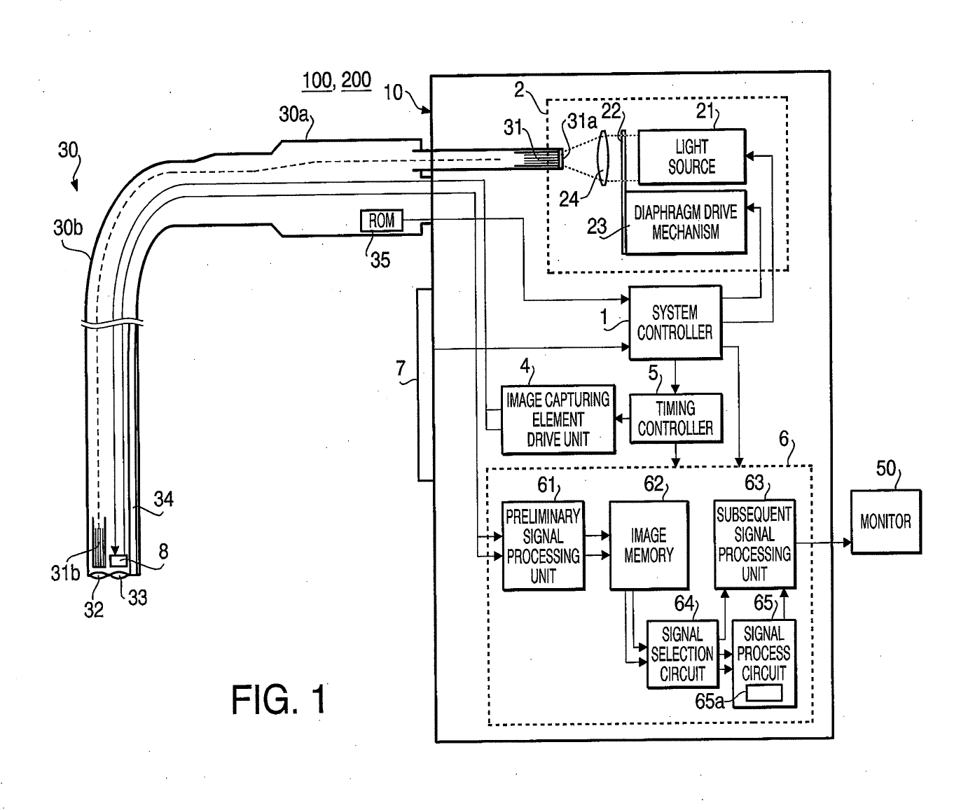

FIG. 1 is a schematic diagram of an endoscope system 100 according to a first embodiment of the present invention. The endoscope system 100 includes a processor 10, an electronic endoscope 30, and a monitor 50. The electronic endoscope 30 is one of a plurality of exchangeable electronic endoscopes, which are specifically designed to be inserted into a body cavity.

The processor 10 is connectable with any of the plurality of exchangeable endoscopes so that each electronic endoscope 30 can be connected to the processor 10. The electronic endoscope 30 is provided with a connecting portion 30a, a flexible tube 30b having an image capturing system at a front end thereof, and a gripper portion (not shown) as an operation system. The electronic endoscope 30 is optically and electrically connected to the processor 10 through the connecting portion 30a. The monitor 50 is connected to the processor 10 to display predetermined information transmitted from the processor 10.

The processor 10 is provided with a system controller 1, a light source unit 2, an image capturing element drive unit 4, a timing controller 5, an image processing unit 6, and a front panel 7.

The electronic endoscope 30 includes a light guide 31, a luminous intensity distribution lens 32, an objective lens 33, an image capturing unit 8, a forceps outlet 34, and a ROM 35. The luminous intensity distribution lens 32, the objective lens 33, and the image capturing unit 8 are arranged in vicinity of the front end of the flexible tube 30b. It is noted that some of the lenses such as the luminous intensity distribution lens 32 and the objective lens 33 are configured to be lens units having a plurality of lenses, however, in the drawings shown herein are indicated as single lenses for simplicity in explanation.

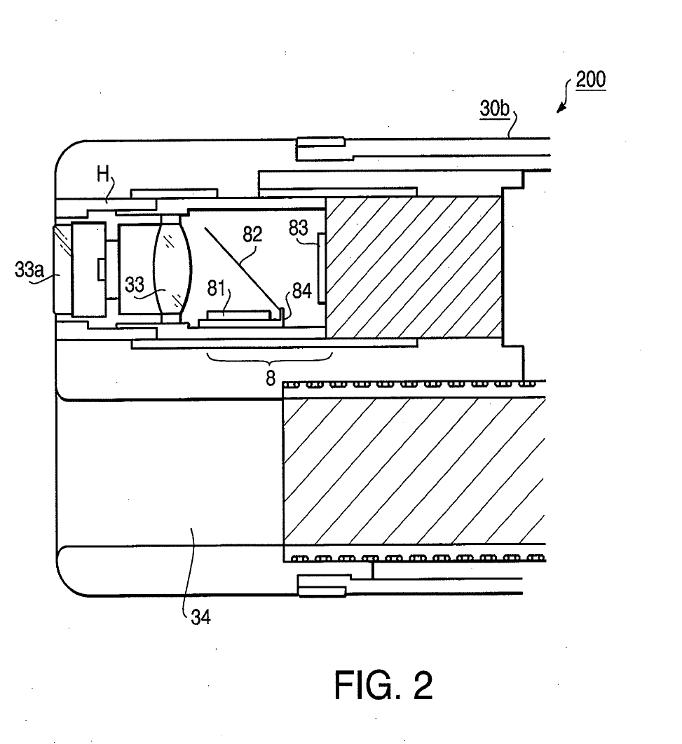

FIG. 2 is a partially enlarged cross-sectional view of the electronic endoscope 30, taken from a plane including an optical axis of the objective lens 33, according to the first embodiment of the present invention. As shown in FIG. 2, the flexible tube 30b has the image capturing unit 8, a cover glass 33a, and the objective lens 33 at the front end thereof. The cover glass 33a, the objective lens 33, and the image capturing unit 8 are arranged inside a cylindrical housing H. The image capturing unit 8 includes a first image capturing element 81, a half mirror 82, a second image capturing element 83, and a supporting member 84. The first image capturing element 81 and the half mirror 82 are fixed to the supporting member 84, which is fixed to the inner periphery of the housing H.

The first image capturing element 81 and the second image capturing element 83 are in a positional relation wherein acceptance surfaces thereof are substantially perpendicular to each other. The half mirror 82 is arranged in a position to make an angle of approximately 45 degrees with respect to the acceptance surfaces of either of the first image capturing element 81 and the second image capturing element 83. In this arrangement, the light from the body cavity received through the objective lens 33 is divided by the half mirror 82 and directed to each of the image capturing elements 81, 83. In the present embodiment, the half mirror 82 is adapted to have light transmission characteristics of 50 percent (i.e., 50 percent of reflection characteristics). Therefore, amounts of the lights received in the image capturing elements 81, 83 are substantially equivalent.

The supporting member 84 that supports the first image capturing element 81 and the half mirror 82 is formed to have an approximate cross-sectional shape of an L, as the cross-section is taken by the plane including the optical axis of the objective lens 33. The supporting member 84 is configured to be movable in a direction parallel to the optical axis of the objective lens 33 in the inner periphery of the housing H. Thus, focusing in the first image capturing element 81 is adjusted as the supporting member 84 is moved. It is noted that the second image capturing element 83 is fixed in a position wherein the light received in the second image capturing element 83 is focused substantially correctly on the acceptance surface, therefore, focusing mechanism is not necessary.

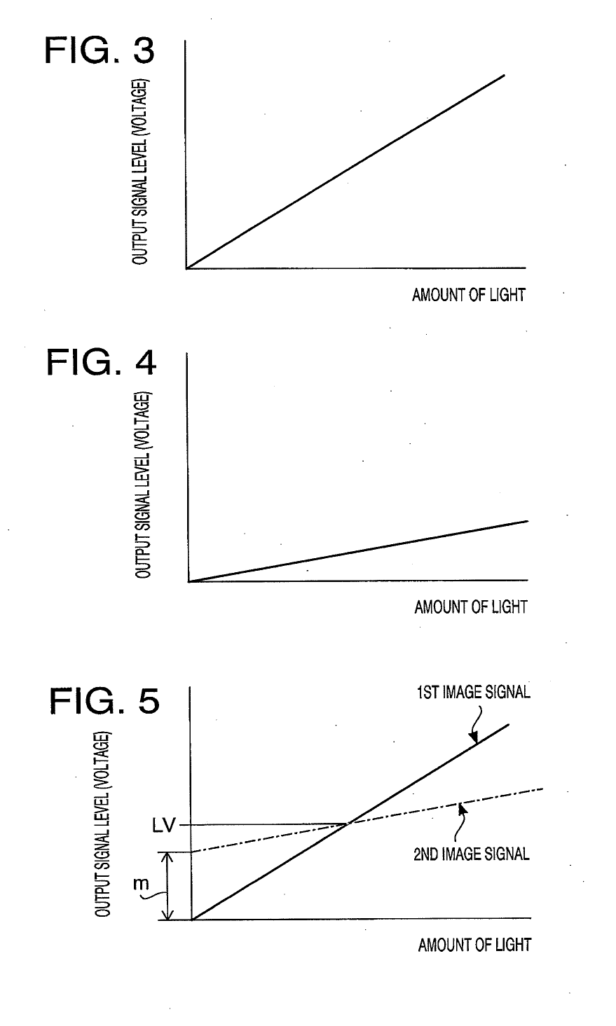

The image capturing elements 81, 83 are color-enabled CCDs (charge-coupled device) having different correspondences between amounts of light and output signal levels. FIG. 3 is a chart showing the correspondence between amounts of light and output signal levels in the first image capturing element 81 according to the first embodiment of the present invention. FIG. 4 is a chart showing the correspondence between amounts of light and output signal levels in the second image capturing element 83 according to the first embodiment of the present invention. It is noted that, as shown in FIGS. 3 and 4, the image capturing element 81 has higher sensitivity than the image capturing element 83 when amount of light to be received is equivalent.

When the endoscope system 100 as described above is used for observing, the front end portion of the electronic endoscope 30 (i.e., the front end of the flexible tube 30b) is arranged in vicinity of the object to be captured. When, for example, the object is in vivo tissue inside a body cavity, the front end portion of the flexible tube 30b is inserted to be closer to the in vivo tissue in the body cavity to capture an image of the observation object. In this position, when the operation unit of the electronic endoscope 30 is operated by an operator, the processor 10 executes predetermined controlling processes.

The system controller 1 controls various processes executed in each part of the endoscope system 100 as well as image capturing operations in the processor 10. In the present embodiment, as the electronic endoscope 30 is connected in the endoscope system 100, the system controller 1 reads data (identifying data) which is uniquely provided to the electronic endoscope 30 being connected, such as an ID (for example, model name and number of the endoscope) and specification (for example, image format of the image capturing element, pixel numbers, gamma characteristics, and correspondence between the light amount and output signal levels), from the ROM 35. Thus, the processor 10 in the present embodiment is configured to obtain usable data for controlling from the endoscope 30 being connected to the processor 10 itself so that a predetermined process for the obtained specific data is performed. It is noted that the processor 10 in the present embodiment is compatible with existing electronic endoscopes as well as the electronic endoscope 30 of the present invention.

The identifying data of the electronic endoscope 30 includes information regarding a threshold, which is used for generating a clear image corresponding to brightness in the observation object. Description of the threshold will be provided hereinbelow.

Meanwhile, the system controller 1 further controls the light source unit 2. The light source unit 2 includes a light source 21, an aperture diaphragm 22, a diaphragm drive mechanism 23, and a collecting lens 24. As controlling signals from the system controller 1 are received in the light source unit 2, light is emitted from the light source 21. In the present embodiment, a known white light source, such as a metal halide lamp, a xenon lamp, and a halogen lamp, is used as the light source 21.

The light emitted from the light source 12 is transmitted through the aperture diaphragm 22 and the collecting lens 24 and enters the light guide 31 from an inlet end 31a. The light guide 31 is composed of a bundle of optical fibers, which transmits the light received at the inlet end 31a therethrough and emits from an outlet end 31b. The light is then transmitted through the luminous intensity distribution lens 32 and emitted from the front end of the flexible tube 30b to be cast on the observation object. The light is then reflected by the observation object and enters the image capturing unit 8 through the cover glass 33a and the objective lens 33.

The light received in the image capturing unit 8 is divided substantially equivalently into two pencils of light by the half mirror 82 to direct the divided light into each of the image capturing elements 81, 83, and each pencil is focused on the acceptance surface of each of the image capturing elements 81, 83 to form an image. The image capturing elements 81, 83 are driven by the image capturing element drive unit 4. More specifically, the image capturing element drive unit 4 being controlled by the system controller 1 transmits driving signals to the image capturing elements 81, 83 in predetermined timing defined by the timing controller 5. The image capturing elements 81, 83 are synchronized to the driving signals provided by the image capturing element drive unit 4 and generate voltage signals (image signals) according to the images formed on the acceptance surfaces thereof. The generated voltage signals are periodically transmitted to the image processing unit 6 of the processor 10. It is noted in FIG. 1 that a signal line connecting the image capturing element drive unit 4 and the image capturing unit 8 is indicated by one line for simplicity in explanation, however, the number of actual signal lines corresponds to a number necessary to drive each image capturing element. In the present embodiment, four signal lines (input signal line and output signal line for each of the image capturing elements 81, 83 respectively) are provided.

The image processing unit 6 in the present embodiment includes a preliminary signal processing unit 61, an image memory 62, a subsequent signal processing unit 63, a signal selection circuit 64, and a signal process circuit 65. The preliminary signal processing unit 61 is adapted to perform various processes such as A/D (analog-to-digital) conversion, gain adjustment, and resolution adjustment, on the voltage signals output from the image capturing elements 81, 83. The voltage signals output from the preliminary signal processing unit 61 are processed to be image data corresponding to one frame, which is respectively associated with the sensitivity of one of the image capturing elements 81, 83 and are respectively stored in each predetermined area of the image memory 62. The image data for one frame stored independently is then output to be image signals to the signal selection circuit 64 in synchronization with the timing signals provided by the timing controller 5. The timing signals are transmitted in correspondence with, for example, monitor intervals. In the present embodiment, the image signal obtained through the image capturing element 81, which has the higher sensitivity, is referred to as first image signal, while the image signal obtained through the image capturing element 82, which has the lower sensitivity, is referred to as second image signal.

The signal selection circuit 64, controlled by the system controller 1, receives the image signals from the image memory 62, selects usable signals between the first image signal and the second image signal, and outputs the usable signals to the subsequent signal processing unit 63 and ultimately to the monitor 50 as an image to be viewed. In the present embodiment, the front panel 7 of the processor 10 is provided with the operation unit (not shown), which is operated by the operator to select a desired image to be displayed on the monitor 50. As the operator selects the desired image to be displayed and operates the operation unit accordingly, instruction signals corresponding to the selection are inputted in the system controller 1. The system controller 1 then controls the signal selection circuit 64 according to the instruction signals received.

With the above configuration, if the operator selects to obtain images which are substantially clear to observe regardless of the brightness differences in the observation object (hereinafter referred to as processed images), for example, the signal selection circuit 64 outputs all the image signals (i.e., the first and the second image signals) obtained from the image memory 62 to the signal process circuit 65. The first and the second image signals are then temporarily stored in a memory area 65a of the signal process circuit 65.

In the present embodiment, the signal process circuit 65 generates usable signals for the processed images. FIG. 5 is a chart showing correspondence between amounts of light and output signal levels in the first and second image capturing elements 81, 83 according to the first embodiment of the present invention. In FIG. 5, the horizontal axis indicates the amounts of light entering the image capturing elements 81, 83 while the vertical axis indicates the signal levels output from the image capturing elements 81, 83. Further, the solid line represents the first image signal while the dash-and-dotted line represents the second image signal. It should be noted that an inclination of the dash-and-dotted line representing the second image signal is shifted vertically for a predetermined correction value m from the inclination indicated in FIG. 4 so that the dash-and-dotted line with a predetermined inclination meets the solid line representing the first image signal at a point where the solid line meets the predetermined level LV. The output level of the second signal is offset by m to a level wherein the output level of the first signal substantially equals the predetermined level LV. Detail of the offset value m will be described later.

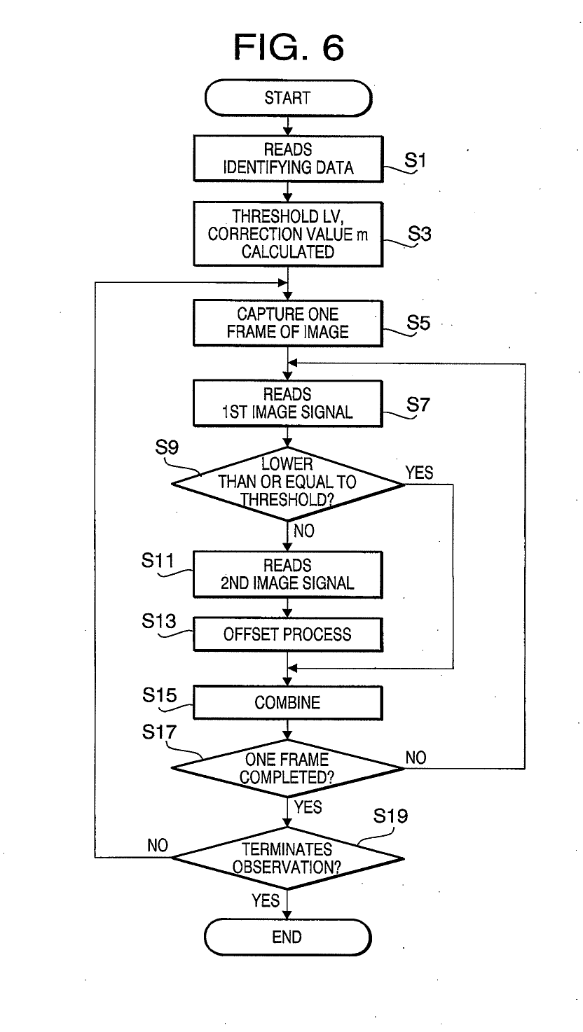

FIG. 6 is a flowchart to illustrate a signal modifying process to be executed by the processor 10 according to the first embodiment of the present invention. Through this process, the signal process circuit 65 generates a usable signal for one frame of an image to be transmitted to the subsequent signal processing unit 63. More specifically, one frame of the image captured by the image capturing elements 81, 83 is divided into a plurality of areas, and image data in each divided area is examined as to whether the image in the area has suitable brightness for observation and modified if necessary so that the white and black balance in the divided image, eventually an overall image, can be suitably maintained.

As the process starts, in S1, the system controller 1 of the processor 10 reads the identifying data including the threshold of the electronic endoscope 30 from the ROM 35. In S3, based on the obtained identifying data, a threshold level LV and a correction value m to be used for generating usable signals are calculated. The threshold level LV is an index to be used when the signal selection circuit 64 selects usable signals between the first image signal and the second image signal and is determined on basis of an electronic endoscope 30 (i.e., on basis of an object to be observed.) The calculate threshold level LV is transmitted to the signal process circuit 65.

Next, in S5, the system controller 1 controls the electronic endoscope 30 to capture an image for one frame. In this step, the first image signal for one frame and the second image signal for one frame are stored in the memory area 65a of the signal process circuit 65.

Next, in S7, the system controller 1 controls the signal process circuit 65 to read the first image signal from the memory area 65a. In the present embodiment, in each divided area, a set of four pixels, which are R (red), G (green), G (green), and B (blue), is provided. Therefore, in S7, the signal process circuit 65 reads the first image signal for the four pixels from the memory area 65a.

Next, in S9, the signal process circuit 65 examines the first image signal for the divided area to judge as to whether an average output level of the read first image signal is lower than or equal to the threshold level LV which was determined in S3. It is noted that in the present embodiment the first image signal being compared with the threshold level LV is substantially equivalent to the voltage level from the image capturing element 81 (i.e., the amount of light entering the image capturing element 81.)

When the average output level of the first image signal is greater than the threshold LV (S9: NO), the process proceed to S11. In S9, it can be considered that brightness in the divided area is high, and details in the area may be lost in white when the image is generated based on the first image signal.

Next, in S11, the second image signal corresponding to the same divided area is read from the memory area 65a. It is noted that in this step the second image signal with the output characteristics shown in FIG. 4, which is obtained through the second image capturing element 83 with lower sensitivity, is obtained to be used in the frame of image so that details (white balance) in the divided area can be effectively maintained.

Next, in S13, the signal process circuit 65 offsets the second image signal obtained in S11 by adding the correction value m. The correction value m is set such that the output level of the second image signal, which corresponds to the amount of light entering the endoscope 30 when the output signal level of the first image signal meets the threshold level LV, is shifted to meet the threshold level LV (i.e., the output signal level of the second image signal is shifted to meet the output signal level of the first image signal at a point where the first image signal meets the threshold level LV.) (See FIG. 5.) Thus, the image signals to be used to configure one frame of image may vary depending on their output signal levels (i.e., brightness) in each divided area. However, it is noted that the brightness is thus adjusted by offsetting the second image signal so that, for example, an image area which is generated based on the second image signal should not be displayed to be darker than the other image areas which are generated based on the first image signals.

Meanwhile, in S9, when the average output level of the first image signal is smaller than or equal to the threshold LV (S9: YES), or following S13, the signal process circuit 65 proceeds to S15, wherein the image signal for the divided area is generated. More specifically, one of the first image signal, when affirmative judgment is made in S9, and the offset second image signal, when negative judgment is made in S9, is used to generate the image signal in the divided area. The generated image signal is then used to compose the frame of image with the other divided areas configuring the frame.

Next, in S17, it is judged as to whether image signals for all the divided area in the frame are generated. If an unprocessed area remains (S17: NO), the process returns to S7 and repeats S7-S17. When affirmative judgment is made in S17 (S17: YES), in S19, it is judges as to whether the operator selects to terminate the observation. If it is judged that the operator does not select termination (S19: NO), the process returns to S5 and repeats S5-S19. If the operator selects to terminate the observation (S19: YES), the signal process circuit 65 transmits the processed signals (usable signals) for the frame to the subsequent signal processing unit 63, and terminates the process.

The subsequent signal processing unit 63 converts the usable signals generated and transmitted from the signal process circuit 65 into analog signals (D/A conversion), and generates video signals, which are compliant with a signal reception standard of the monitor 50. As the monitor 50 receives the video signals from the subsequent signal processing unit 63, images corresponding to the video signals are displayed frame by frame in the monitor 50 accordingly.

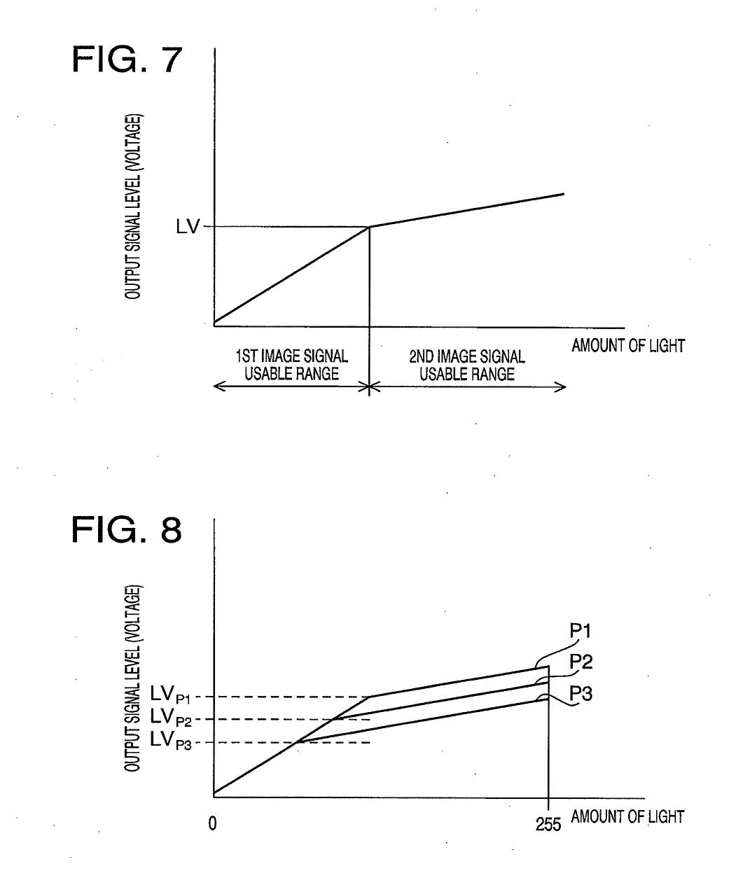

FIG. 7 is a chart showing correspondence between the amount of light and the output signal level of the usable signals generated by the signal process circuit 65 according to the first embodiment of the present invention. As shown in FIG. 7, the usable signals have characteristics of the two image signals provided from the image memory 62 which are selectively used depending on the amount of light entering the image capturing elements 81, 83. Thus, a clear image, responsively obtained from the usable signals depending on the amount of light in the observation object, maintaining details, which may otherwise be lost in white or black, can be displayed in the monitor 50.

In the present embodiment, the endoscope system 100 is capable of having a plurality of electronic endoscopes 30 which can be exchanged for various observing conditions. As previously described above, each electronic endoscope 30 is provided with its unique information, including the threshold level LV and the correction value m, to be used for preferable operations. FIG. 8 is a chart showing correspondence between amounts of light and output signal levels in three image capturing elements according to the second embodiment of the present invention. In FIG. 8, P1 indicates a characteristic of a processed signal which is used when an object having more dark areas (e.g., large intestines) is observed. P3 indicates a characteristic of a processed signal which is used when an object having more bright areas (e.g., stomach) is observed. P2 indicates a characteristic of a processed signal which is intermediate between P1 and P3.

As shown in FIG. 8, a threshold level LV_{P1}, for P1 is greater than LV_{P2}, which is a threshold level for P2, and LV_{P2} is greater than LV_{P3}, which is a threshold level for P3 (LV_{P3}<LV_{P2}<LV_{P1}.) Thus, when it is expected that an object for the electronic endoscope 30 has brighter areas, the threshold level LV is set to be lower so that the white balance in the captured image can be maintained and the brighter areas can be displayed clearly in the image. On the other hand, when it is expected that an object for the electronic endoscope 30 has darker areas, the threshold level LV is set to be higher so that the details should not be lost in black and the darker areas can be displayed clearly in one frame of the image.

In FIG. 8, it is noted that the characteristics P1-P3 are schematically indicated. Therefore, for example, the output signal levels when the amount of light is a maximum level (e.g., 255 in a 256 color scale) are different in each of P1-P3 (i.e., in each electronic endoscope 30 to be used.) However, in actual use, the output signal levels are corrected through a gamma correction process in the subsequent signal processing unit 63 so that the images ultimately displayed on the monitor 50 can be properly provided regardless of individual specifics of the electronic endoscopes 30. Thus, for example, when amount of light is at a maximum level, the output signal level is also at its maximum.

In the present embodiment, at least ones of the images captured through the image capturing element 81 and the images captured through the image capturing element 83 can be selectively displayed on the monitor 50. In this case, one frame of image being displayed is generated based on the selected image signal (i.e., the first image signal or the second image signal) which is not processed or composed in the signal process circuit 65. If the operator selects to the unprocessed images to be displayed, the signal selection circuit 64 outputs the image signals corresponding to the selection directly to the subsequent signal processing unit 63. Further, the unprocessed images can be displayed together with the processed images simultaneously displayed in the monitor 50 or solely without the processed images.

Although an example of carrying out the invention has been described above, the present invention is not limited to the above described embodiment.

For example, in the first embodiment described above, the amount of light entering the image capturing elements 81, 83, which have different sensitivities, is substantially equivalent. However, it can be configured such that the light entering two image capturing elements, which have substantially equivalent sensitivities, are different.

For another example, in the first embodiment, the image capturing unit 8 is provided with two image capturing elements 81, 83 having different sensitivities, however, the image capturing unit 8 may be provided with three or more image capturing elements having different sensitivities. It is desirable that the number of the image capturing element is determined based on process loads for generating the processed images and clearness of the images desired.

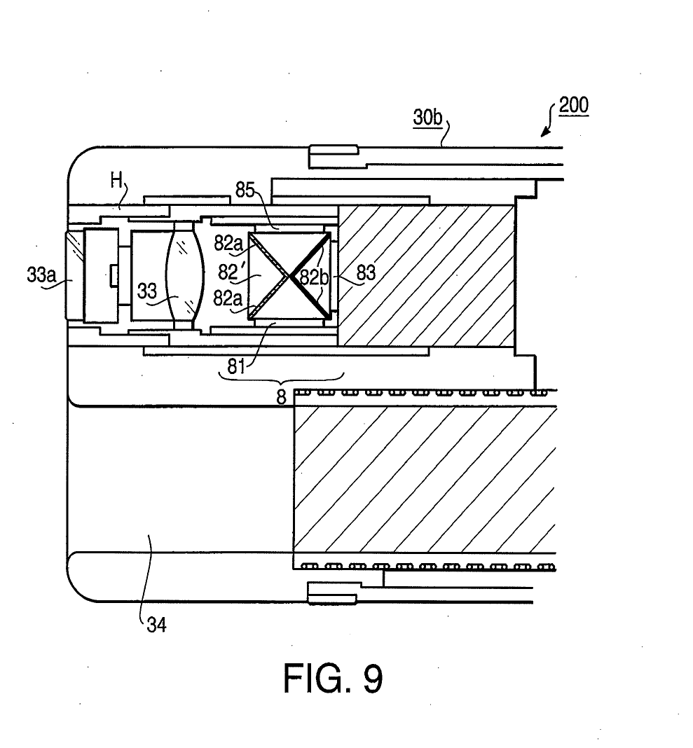

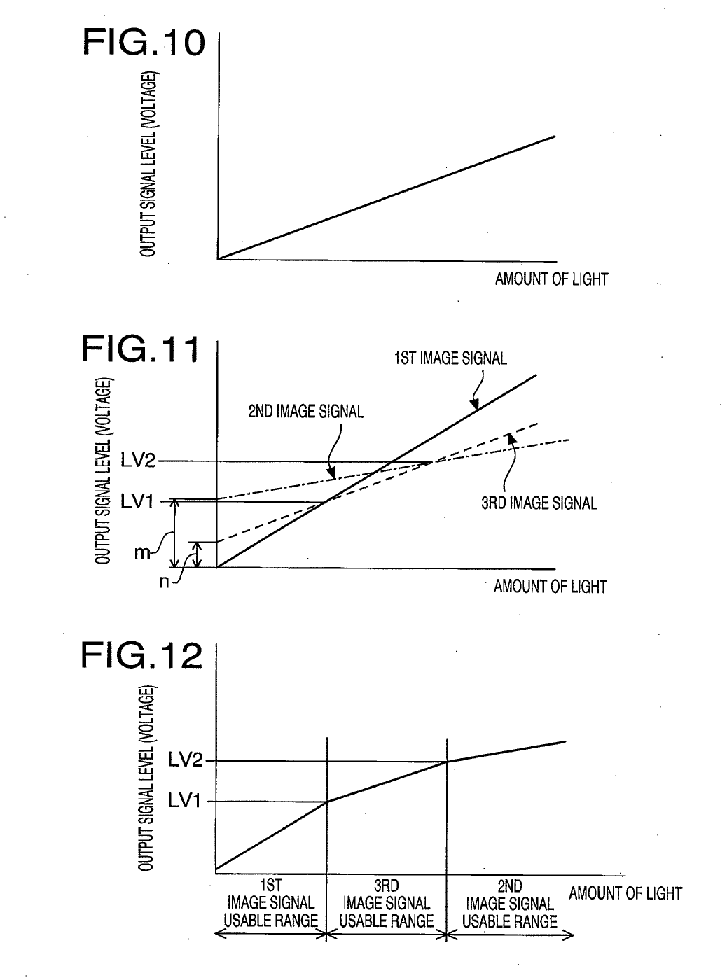

FIG. 9 is a cross-sectional partial view of an electronic endoscope 30b in an endoscope system 200 with three image capturing elements having different sensitivities according to a second embodiment of the present invention. In this embodiment, a configuration similar to that of the first embodiment is referred to by an identical reference numeral, and description of that will be omitted. The image capturing unit 8 is provided with a beam splitter prism 82′ and a third image capturing element 85, in addition to the first and second image capturing elements 81, 83. FIG. 10 is a chart showing correspondence between amount of light and output signal level in the third image capturing element 85 according to the second embodiment of the present invention. As shown in FIG. 10, the third image capturing element 85 has output signal level characteristics for amounts of light, which are in the middle between the output signal level characteristics of the first image capturing element 81 and the output signal level characteristics of the second image capturing element 83. In this configuration, the endoscope system 200 is provided with three signal lines, each of which corresponds to the image capturing elements 81, 85, 83, between the image capturing element drive unit 4 and the image capturing unit 8, and between the image capturing unit 8 and the image processing unit 6.

The beam splitter prism 82′ is configured with four right angle prisms, which are coupled to one another to substantially form a cube. Bonded planes 82a, 82b of the right angle prisms, which substantially form a shape of an X, are provided with optical films to transmit and reflect the incoming beam in a predetermined ratio. In the present embodiment, the bonded planes 82a, 82b have light transmission characteristics (i.e., reflection characteristics), in which amounts of the light entering the image capturing elements 81, 85, 83 are substantially equivalent. More specifically, the bonded plane 82a, which is closer to the objective lens 33 than the bonded plane 82b, has the light transmission ratio wherein one third of the amounts of light is reflected and the remaining two third is transmitted. Further, the bonded plane 82b, which is farther from the objective lens 33, has the light transmission ratio wherein the reflected amount and the transmitted amount of light are substantially equivalent. It is noted that in the present embodiment internal reflection of the beams which are once reflected in the bonded planes 82a, 82b are not taken into consideration for the transmission and reflection ratio.

FIG. 11 is a chart showing correspondence between amount of light and output signal level of usable signals generated by the signal process circuit 65 according to the second embodiment of the present invention. In FIG. 11, the horizontal axis indicates the amounts of light entering the image capturing elements 81, 83, 85, while the vertical axis indicates the signal levels output from each of the image capturing elements 81, 83, 85. Further, the solid line indicates the first image signal, the dash-and-dotted line indicates the second image signal (more specifically, the second image signal having the characteristic shown in FIG. 4, shifted with the predetermined correction value m), and the broken line indicates third image signal output from the third image capturing element 85 (more specifically, the third image signal having the characteristic shown in FIG. 10, shifted with the predetermined correction value n.)

A process to generate the usable signals from the first, the second and the third image signals is substantially similar to the process described in the previous embodiment, therefore, detailed description of that will be omitted. FIG. 12 is a chart showing correspondence between amounts of light and the signal levels of the usable signals generated in the signal process circuit 65 according to the second embodiment of the present invention. As shown in FIG. 12, the usable signals are generated based on the image signals inputted through one of the image capturing elements 81, 83, 85 depending on the amount of light entering the image capturing elements 81, 83, 85. It is noted that in the present embodiment the usable signals are combined with the characteristics of the first, the second, and the image capturing elements 81, 83, 85 and more finely corresponding to the amount of light can be generated. Thus, clear images obtained from the selectively usable signals, maintaining details, which may otherwise be lost in white or black, can be displayed in the monitor 50.

In the endoscope systems 100, 200 according to the embodiments, the threshold level LV can be set to the electronic endoscope 30 individually depending on an object to be observed. It is noted that the endoscope systems 100, 200 can be configured such that the threshold level LV is adjusted by the operator through the operation unit so that operability of the endoscope systems 100, 200 can be improved.

Although examples of carrying out the invention have been described above, the present invention is not limited to the above described embodiment.

For example, it is noted that, in the above embodiments, one frame of the image is divided into a plurality of areas, each of which is configured with four pixels. However, the frame may not necessarily be divided by the four pixels, but may be divided by one scanning line. Moreover, if an improved processing speed of the processor 10 is required, one frame of image may not be divided into a plurality of areas. Instead, an average output level of the first image signal for the entire frame is examined to be compared with the threshold level LV.

For another example, in the first embodiment, the image capturing elements 81, 83 with different sensitivities are used in the endoscope system 100. However, two image capturing elements with substantially equivalent sensitivities can be used. In such a case, for example, the image capturing element drive unit 4 may adjust each of the image capturing elements to have different gain and/or shutter speed. Thus, the output signal level characteristic of one image capturing element and the output signal level characteristic of the other image capturing element can be configured to be different from each other.

Further, a single image capturing element instead of a plurality of image capturing elements can be used. In such a configuration, a signal from the image capturing element can be provided several processes (e.g., various leveled gain adjustments) so that a plurality of image signals with different output levels can be generated. The image signals generated as above can be then used to generate the usable signal.

Furthermore, in the above embodiments, the information including the threshold level LV and the correction value m which is previously determined based on information specific to the electronic endoscope 30 and stored in the ROM 35. However, the operator may input specific information of the electronic endoscope 30 through an input device such as a keyboard, and the processor 30 can determine the threshold level LV and the correction value m based on the inputted information. In this configuration, the electronic endoscope 30 may not necessarily have the ROM 35.

The present disclosure relates to the subject matters contained in Japanese Patent Applications No. P2006-198559, filed on Jul. 20, 2006, and No. P2007-186827, filed on Jul. 18, 2007, which are expressly incorporated herein by reference in their entireties.

What is claimed is:

1. An electronic endoscope system, comprising:
at least one exchangeable electronic endoscope to capture an image of an observation object; and
a processor, which is connectable with the electronic endoscope, to regenerate the image captured by the electronic endoscope,
wherein the electronic endoscope includes:
an optical objective system;
a first image capturing element with higher sensitivity, which captures the image of the observation object and generates a first voltage signal representing the image based on the higher sensitivity;
a second image capturing element with lower sensitivity being lower than the sensitivity of the first image capturing element, which captures the image of the observation object and generates a second voltage signal representing the image based on the lower sensitivity; and
wherein the processor includes:
a threshold setting system, which sets a threshold for judging which of the first voltage signal and the second voltage signal is used to regenerate the image based on suitability of the electronic endoscope;
a judging system, which judges as to whether the first voltage signal obtained from the first image capturing element is either lower than or equal to the threshold set by the threshold setting system; and
an image processing system, which generates a usable signal to be used to regenerate the image based on the first voltage signal when the first voltage signal is judged to be either lower than or equal to the threshold and generates the usable signal based on the second voltage signal when the first voltage signal is judged to be higher than the threshold.

2. The electronic endoscope system according to claim 1,
wherein the electronic endoscope further includes a memory system, which stores information specific to the electronic endoscope; and
wherein the threshold setting system reads the information from the memory system of the electronic endoscope being connected with the processor so that the threshold is set based on the information.

3. The electronic endoscope system according to claim 2, wherein the information specific to the electronic endoscope includes information concerning the threshold.

4. The electronic endoscope system according to claim 3, comprising a plurality of the exchangeable electronic endoscopes,
wherein each of the plurality of exchangeable electronic endoscopes is provided with the information concerning the threshold which is specific to the exchangeable electronic endoscope.

5. The electronic endoscope system according to claim 1,
wherein the image processing system divides one frame of the image captured by the electronic endoscope into a plurality of divided areas, generates the usable signal for each divided area based on the first voltage signal when the first voltage signal is judged to be lower than or equal to the threshold and on the second voltage signal when the first voltage signal is judged to be higher than the threshold, and combines the generated usable signals for each divided area to regenerate one frame of the image based on the generated usable signal.

6. The electronic endoscope system according to claim 1,
wherein the image processing system adjusts the second voltage signal by adding a correction value to the second voltage signal so that an output level of the second voltage signal substantially equals an output level of the first voltage signal at a point where the output level of the first voltage signal substantially equals the threshold; and
wherein the correction value is determined based on the suitability of the electronic endoscope.

7. The electronic endoscope system according to claim 1,
wherein the electronic endoscope is provided with a beam splitter to split a beam entering the electronic endoscope substantially evenly into two directions so that substantially same amount of light is transmitted to the first image capturing element and the second image capturing element; and
wherein the beam splitter includes a half mirror, which deflects a part of the beam entering the electronic endoscope toward the first image capturing element and transmits the remaining part of the beam to the second image capturing element.

8. The electronic endoscope system according to claim 7,
wherein the first image capturing element and the half mirror are integrally attached to a driving system, and
wherein the first image capturing element and the half mirror are driven by the driving system which is slidable in parallel with an optical axis of the objective optical system so that focusing in the first image capturing element is adjusted.

9. The electronic endoscope system according to claim 1,
wherein the image processing system generates a first image signal based solely on the first voltage signal obtained from the first image capturing element and generates a second image signal based solely on the second voltage signal obtained from the second image capturing element.

10. The electronic endoscope system according to claim 9, comprising
a displaying device, which displays the image regenerated by the processor on a screen,
wherein the displaying device is capable of simultaneously displaying at least one of an image corresponding to the usable signal, an image corresponding to the first image signal, and an image corresponding to the second image signal on the screen.
